(19) United States
(12) Patent Application Publication
(10) Pub. No.: US 2023/0066416 A1
(43) Pub. Date: Mar. 2, 2023

(54) Test kit for detecting acetylcholinesterase inhibitors

(71) Applicant: Dr. Franz Köhler Chemie GmbH, Bensheim (DE)

(72) Inventors: Gernot Köhler, Alsbach-Hähnlein (DE); Frauke Weiss, Seeheim-Jugenheim (DE)

(21) Appl. No.: 17/893,464

(22) Filed: Aug. 23, 2022

(30) Foreign Application Priority Data
Aug. 24, 2021 (DE) 202021104545.0

(51) Int. Cl. C12Q 1/46; B01L 9/06

(52) U.S. Cl. CPC C12Q 1/46; B01L 9/06

(57) ABSTRACT

A test kit for detecting organophosphate compounds, includes a support apparatus, a sampling device, a buffer solution in a sample tube, and an acetylcholinesterase as the first active component and a mixture of 5,5′-dithiobis(2-nitrobenzoic acid) (DTNB) and acetylthiocholine. The sampling device is packaged in a sample tube, where the first active component is applied in a first reagent cap and the second active component is applied in a second reagent cap, and the first and second reagent caps each seal a sample tube. The support apparatus has at least four fixing devices for receiving the sample tubes. The sample tubes are fixed parallel to the upper side of the support apparatus, and the support apparatus has at least two receiving devices for receiving the first and second reagent caps and one receiving device for vertically receiving a sample tube.

### CROSS-REFERENCE TO RELATED APPLICATIONS

This application claims the benefit of foreign priority of German Patent Application No. 20 2021 104 545.0, filed Aug. 24, 2021, the entire disclosure of which is incorporated herein by reference.

### FIELD OF THE INVENTION

The invention relates to a test kit for detecting acetylcholinesterase inhibitors. In particular, the invention relates to a mobile test kit for detecting acetylcholinesterase inhibitors by means of a color reaction and visual detection.

### BACKGROUND OF THE INVENTION

Known examples of acetylcholinesterase inhibitors are, for example, the phosphoric acid esters, tabun, soman, sarin, VX, cyclosarin, or Novichok. They affect the human organism by binding to the enzyme acetylcholinesterase (AChE) and thus disrupting its function. As a result, the neurotransmitter acetylcholine can no longer be broken down, resulting in an excess of acetylcholine in the synaptic cleft. This leads to overstimulation of the cholinergic system and thus to a variety of physical symptoms such as constricted pupils, hypersalivation, bradycardia, convulsions, and respiratory depression. Depending upon the severity of poisoning, it can result in death by asphyxiation.

Rapid detection of a corresponding poisoning is crucial here both for initiating countermeasures for the poisoning victim and for detecting contamination in order to avoid corresponding poisoning. There is therefore a need for mobile test kits allowing rapid and reliable detection on-site.

A detection reaction is known from the prior art, in which detection takes place indirectly and visually via a color reaction. Here, a sample is first taken and mixed with human acetylcholinesterase, hereafter also referred to as hAChE, in a buffer solution. A mixture of acetylthiocholine and 5,5′-dithiobis(2-nitrobenzoic acid) (DTNB), also known as Ellman's reagent, is then added to the reaction solution. In the presence of an active acetylcholinesterase, acetylthiocholine is converted to thiocholine, which in turn reacts with 5,5′-dithiobis(2-nitrobenzoic acid) to release 2-nitro-5-thiobenzoate (TNB⁻). In base and neutral solutions, 2-nitro-5-thiobenzoate (TNB⁻) deprotonates to the yellow TNB²⁻. This has a characteristic yellow color so that the test solution has a yellow color of varying intensity depending upon the concentration of the released 2-nitro-5-thiobenzoate (TNB⁻). On the other hand, if the DTNB is not broken down because the added acetylcholinesterase is inhibited and no thiocholine is formed, the solution remains colorless. Using a color table, the user can thus determine whether an acetylcholinesterase inhibitor is present in the sample taken.

In order to ensure rapid detection and, if necessary, decontamination, rapid detection of acetylcholinesterase inhibitors that can be carried out on-site is necessary. Mobile test kits are known from the prior art, with which acetylcholinesterase inhibitors can be detected on-site in accordance with the principle described above. These kits comprise several individual components, which are usually heat-sealed in a foil bag to protect them from environmental influences. As an individual component, the kit contains a cotton swab for sampling, which is additionally shrink-wrapped in a bag made of aluminum composite foil.

The test kit also contains a sample tube with a buffer solution, a cap into which acetylcholinesterase has been applied, and a cap containing the substances acetylthiocholine and DTNB. The two caps are also additionally packed in a sealed foil bag. The disadvantage of the test kits, however, is that they are confusing due to the large number of individual components and, in addition, opening the individual, heat-sealed foil bags to remove the individual components is time-consuming and also requires certain motor skills, which may not be possible—in particular, if the user is wearing appropriate protective equipment. Furthermore, carrying out the detection is error-prone due to the individual components being present in the outer packaging in an unordered manner. A further disadvantage of the known test kit is the use of aluminum composite foils since these cannot be autoclaved or can be autoclaved only to a limited extent. The bag material can stick together during autoclaving so that the individual components cannot be removed or can be removed only with great difficulty. In order to circumvent this problem, plastic bags suitable for autoclaving are used in other test kits. However, since these have a higher water vapor permeability than the aluminum foils, the shelf life of the test kit is reduced.

### OBJECT OF THE INVENTION

The object of the invention is to provide a mobile test kit for detecting acetylcholinesterase inhibitors, which is free of the disadvantages known from the prior art. In particular, the object of the invention is to provide a corresponding mobile test kit which is easy to handle and at the same time has a long shelf life. Surprisingly, this object is already achieved by the subject matter of the independent claim. Advantageous embodiments and further developments are the subject matter of the dependent claims.

### SUMMARY OF THE INVENTION

The test kit for detecting acetylcholinesterase inhibitors comprises a support apparatus, a sampling device, a buffer solution in a sample tube with a cap, and acetylcholinesterase as a first reactive component and a mixture of acetylthiocholine and DNTB as a second reactive component.

The first and second reactive components are each in solid form and are each applied in a reagent cap that is suitable for sealing the sample tube with the buffer solution. The reagent caps with the first and second reactive components each seal a sample tube. The sample tubes here serve to protect the reactive components applied in the reagent caps. The sample tubes prevent the substances from falling out and contamination of the reactive components. Having the reagent caps screwed onto a sample tube offers several advantages over foil packaging. Unscrewing the cap from the sample tube is easier than opening heat-sealed foil packaging and removing the reagent cap from the opened foil packaging, so that the test kit according to the invention is easier to handle. This applies in particular when protective gloves are used, as this limits the user's fine motor skills.

According to one embodiment of the invention, the sample tube with the reagent cap of the first reactive component and/or the sample tube with the reagent cap of the second reactive component contains a desiccant—preferably silica. In this case, the desiccant is preferably repackaged—for example, in a bag. The desiccant can effectively protect the reagents from environmental influences such as high humidity, which has a beneficial effect on the shelf life of the test kits.

In the test kit according to the invention, the sampling device, e.g., a cotton swab, is also not shrink-wrapped in foil packaging but is packed in a sample tube with a cap. As already described above, this leads to a significantly simplified opening of the outer packaging of the sampling device compared to foil packaging. The sampling device can be removed from the sample tube by gently shaking it. In addition, a sample tube is significantly more stable than foil packaging, so that damage to the outer packaging by an edge of the sampling device, for example, is avoided. Furthermore, during production, the sampling device can be inserted into a tube more easily than shrink-wrapped in foil, so that the use of a sample tube as outer packaging is also advantageous with regard to the manufacture of the test kit.

A further advantage of using sample tubes as outer packaging for the individual components is that, unlike foil bags, no heat-sealing is necessary. This simplifies the manufacture of the test kit. In addition, the sample tubes used can be autoclaved and at the same time have low water vapor permeability, which has an advantageous effect on the shelf life and the permissible period of use of the test kit. Furthermore, sample tubes are easier to position for autoclaving than foil bags, which also simplifies the autoclaving process. According to preferred embodiments, the sample tubes consist, for example, of a plastic such as polypropylene, polyethylene, polystyrene, or polycarbonate.

According to an advantageous embodiment of the invention, the sample tubes have a screw thread, and all the caps are designed as screw caps. This allows a particularly simple, controlled opening and closing of the sample tubes.

The individual components of the test kit are fixed by the support apparatus of the test kit. This allows a clear arrangement of the individual components, and thus simplifies carrying out the test. As a result of the fixed arrangement of the individual components in the support apparatus, the test kit can be taken out of the outer packaging as a whole, and the individual components are prevented from falling. Furthermore, the test kit is arranged very clearly thanks to the support apparatus and allows the detection reaction to be carried out in an orderly manner even in stressful or confusing operational situations. In addition, the support apparatus also serves as a storage place for the individual components while the detection is being carried out, so that the user can easily carry out the detection even when space is limited.

The support apparatus here has an upper side and an underside. At least four fixing devices for fixing the sample tubes of the test kit described above are arranged on the upper side of the support apparatus. The fixing devices are formed by recesses in the support apparatus. The individual fixing devices are preferably arranged parallel to one another and in a row. In this case, the sample tubes are fixed parallel to one another and horizontal to the upper side of the support apparatus. The fixing devices are designed so that the closed end of the sample tube is inserted into the fixing devices first. Thus, the caps of the sample tubes are easily accessible, and can be unscrewed without having to remove the sample tube from the fixing device. This is particularly advantageous for removing the reagent caps with the first and second reactive components.

The positioning of the sample tubes parallel to one another and horizontal to the upper side of the support apparatus is also advantageous since the test kit can be easily laid down during use. In addition, a relatively flat test kit with a low space requirement can be obtained in this way. A low space requirement for the test kit is particularly advantageous if the test kit is carried during use as part of a set of equipment and/or on the body. In addition to requiring little space, a flat test kit also offers the advantage that deformation of the outer packaging and the labels thereon can be avoided, thus ensuring that the labels are easy to read. This is particularly advantageous since, according to one embodiment of the invention, the instructions diagram for carrying out the detection, the color scale for evaluating the test, and/or other relevant information can be applied in the form of at least one label to the outer packaging of the test kit.

Furthermore, the support apparatus has on the upper side at least two receiving devices for receiving the caps of the sample tubes and one receiving device for vertically receiving a sample tube. The receiving devices are designed as depressions in the upper side of the support apparatus. The support apparatus preferably has a closed underside.

The receiving devices for receiving the reagent caps, also referred to as cap holders, are preferably circular in shape and have a diameter that is larger than the diameter of the reagent caps. The reagent caps with the reactive components can be placed or stored in the cap holders. In addition to providing a clearer arrangement, this also offers the possibility of visualizing the detection steps that have already taken place, and thus reduces the susceptibility to error in the detection due to usage errors—particularly in stressful and/or time-critical operational situations. One embodiment of the invention provides that the depth of the recess h₁ in the surface that forms the cap holder be smaller than the thickness of the support holder. This prevents the cap from slipping through the support holder. Alternatively or additionally, the depth of the recess h₁ is smaller than the height of the cap, so that only part of the cap is held by the cap holder. This makes it easier to remove the reagent cap from the cap holder if, for example, the cap holder is used only to temporarily store the cap.

The receiving device for vertically receiving a sample tube, also referred to as a sample holder, has the same cross-sectional shape as the sample tube, and in particular as the sample tube with the buffer solution. Here, the diameter and depth of the cutout in the support apparatus is selected such that the sample tube can be placed in the corresponding receiving device. In order to ensure that the sample tube is received securely in the receiving device, it is particularly advantageous if the depth h₂ of the device is greater than the depth h₁.

However, the depth of the recess is preferably less than the overall thickness of the support apparatus. The receiving device for vertically receiving a sample tube thus fulfills the function of a sample holder and makes it possible to safely place the sample tube with the buffer or reaction solution in the support apparatus between the individual detection steps. This means that the user has both hands free to carry out the individual detection steps, which considerably simplifies carrying out the detection. In addition, the risk of accidentally spilling the buffer or reaction solution is significantly reduced.

It has proven to be advantageous for carrying out the detection if the sample holder and the cap holders are arranged in a row on the support apparatus. Thanks to the clear arrangement achieved in this way, usage errors can be further minimized.

According to one embodiment, the support apparatus is designed in one piece. As a result, the stability of the support apparatus can be improved. A one-piece support apparatus can also be advantageous with regard to the manufacturing process since joining processes can be dispensed with, for example. The support apparatus preferably comprises or consists of plastics, such as acrylonitrile butadiene styrene (ABS), polylactate (PLA), polypropylene (PP), polystyrene (PS), polycarbonate (PC), or polyethylene (PE). According to a particularly advantageous embodiment, the support apparatus is an injection-molded part or a 3-D print.

The fixing devices for receiving the sample tubes can in particular be clamping or plug-in devices. According to a preferred embodiment, the fixing devices are designed as clamping devices and have a cross-section in the shape of a circular arc. The sample tube can be fixed particularly well in the fixing device if the arc length of the cross-section is greater than half the circumference of the corresponding circle circumference. In this embodiment, the sample tube is partially surrounded by the fixing device. In this embodiment, the individual fixing devices are preferably arranged in a row, and, between the individual fixing devices, the support apparatus has cutouts on the upper side, wherein the cutouts preferably have the same length as the fixing devices. The recesses preferably have an angular cross-section, and particularly preferably a rectangular cross-section. As a result of the recesses between the individual fixing devices, these have relatively thin lateral parts. These lateral parts give the fixing device a certain flexibility in its structure so that it can be widened in places by the sample tubes. A sample tube can thus be securely clamped in the fixing device and removed again by applying little force.

According to one embodiment, the sample tube with the buffer solution contains a buffer solution with a pH in the range of 6 to 8, and preferably in the range of 7.3 to 7.5.

In particular, the buffer is a phosphate buffer.

The cap with the first reactive component contains acetylcholinesterase (AChE), and preferably human acetylcholinesterase (hAChE). The use of human acetylcholinesterase has the advantage that all acetylcholinesterase inhibitors relevant to the human organism can be detected. Thus, not only organophosphates acting on the cholinogenic system, but also, for example, carbamates can be detected. The test kit is therefore designed or equipped in particular to detect a nerve agent, such as sarin, tabun, VX, mustard gas, soman, cyclosarin and/or Novichok, or a pesticide as an acetylcholinesterase inhibitor or another substance that inhibits acetylcholinesterase.

### BRIEF DESCRIPTION OF THE DRAWINGS

The invention is described in more detail below with reference to FIGS. 1 through 11. Shown are:

FIG. 1 a schematic representation of the detection reaction of acetylcholinesterase inhibitors with the help of the test kit,

FIG. 2 a schematic representation of the test kit in plan view,

FIG. 3 a schematic representation of the support apparatus in plan view,

FIG. 4 a schematic representation of a cross-section through the partial region of a support apparatus having a cap holder and sample holder,

FIG. 5 a schematic representation of a cross-section through the partial region of a support apparatus having fixing devices,

FIG. 6 a schematic representation of a cross-section through the partial region of a support apparatus having a cap and sample tube,

FIG. 7 a photograph of an outer packaging of a test kit as an embodiment,

FIG. 8 a photograph of a test kit as an exemplary embodiment,

FIG. 9 a photograph of a test kit, wherein the test kit was prepared for performing the detection reaction,

FIG. 10 a photograph of a packaged test kit from the prior art, and

FIG. 11 a photograph of the individual components of the test kit shown in FIG. 10.

### DETAILED DESCRIPTION OF THE INVENTION

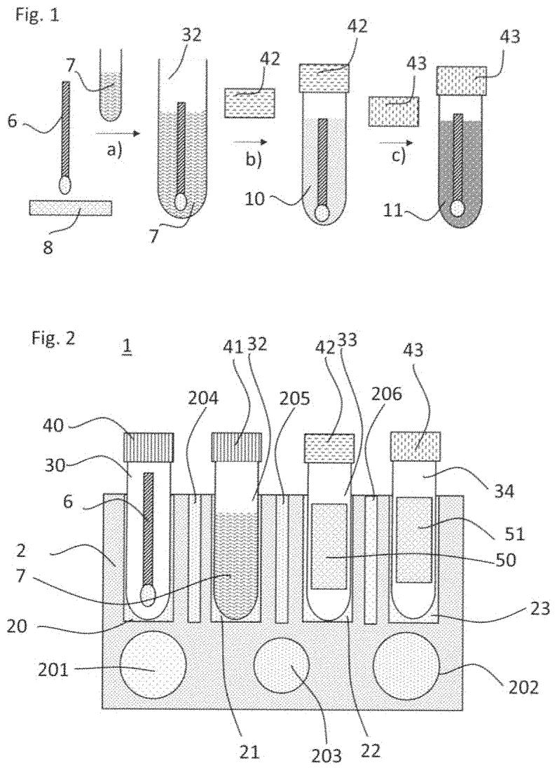

FIG. 1 is a schematic representation of the performance of the test for acetylcholinesterase inhibitors—in particular, a nerve agent, such as sarin, tabun, VX, mustard gas, soman, cyclosarin and/or Novichok, a pesticide or another substance that inhibits acetylcholinesterase—with the test kit according to the invention. In a first step a), a swab is taken of the surface 8 to be examined, using the sampling device 6, e.g., a cotton swab, and the sampling device 6 is placed in a sample tube 32 with a buffer solution 7.

Subsequently, in step b), the reagent cap 42 is screwed onto the sample tube 32. Acetylcholinesterase is located in the reagent cap 42 as the first reactive component.

The sample tube 32 is then shaken for 10 seconds, and thus both the AChE inhibitor possibly absorbed by the sampling device and the acetylcholinesterase present in the cap 42 are brought into the buffer solution such that the sample solution 10 is formed.

After a waiting time of one minute, within which the AChE inhibitor optionally present can bind to AChE and thus completely or partially inhibit it, in step c), the reagent cap 42 is replaced by the reagent cap 43. DTNB and acetylthiocholine are located in the reagent cap 43 as the second reactive component. In order to dissolve the second reactive component out of the reagent cap 43, the sample tube 32 is shaken again, so that the sample solution 10 can dissolve the components out of the reagent cap 43.

Subsequently, the coloring of the solution 11 thus obtained is compared with a color chart. A yellow coloration of the solution 11 is caused by the TNB2− ion and indicates the presence of an active, i.e., non-inhibited, acetylcholinesterase in solution 11. A yellow coloration is therefore an indicator of the absence of an acetylcholinesterase inhibitor in sample 8, and thus represents a negative result for acetylcholinesterase inhibitors. If, on the other hand, the sample 8 contains acetylcholinesterase inhibitors, a yellow coloration of the solution 11 remains in step c), or only a very weak yellow coloration of the solution 11 can be observed.

FIG. 2 is a schematic representation of the test kit 1 in plan view. The test kit 1 comprises the support apparatus 2, as well as the sample tubes 30, 32, 33, 34 having the caps 40, 41 and the reagent covers 42, 43. The sample tubes 30, 32, 33, 34 are fixed in the fixing devices 20, 21, 22, 23. The fixing devices 20, 21, 22, 23 represent recesses on the upper side 18 (FIG. 5) of the support apparatus 2 and are arranged parallel to one another in a row. In the embodiment shown in FIG. 2, the fixing devices 20, 21, 22, 23 are designed as clamping devices. In order to ensure the flexibility of the fixing devices 20, 21, 22, 23 that is necessary for clamping the sample tubes 30, 32, 33, 34, the support apparatus 2, between the fixing devices 20, 21, 22, 23, has recesses 204, 205, 206 on the upper side 18 of the support apparatus 2.

The sample tubes 30, 32, 33, 34 are fixed parallel to one another and horizontally with respect to the upper side 18 of the support apparatus by means of the fixing devices 20, 21, 22, 23. In this case, the sample tubes 30, 32, 33, 34 are only partially fixed in the fixing devices 20, 21, 22, 23. While the lower part of the sample tubes 30, 32, 33, 34 is located within the fixing devices, the upper part of the sample tubes 30, 32, 33, 34 having the caps 40, 41 and the reagent caps 42, 43 is freely accessible such that removal of the sample tube is not absolutely necessary for removing the caps 40, 42, 43. The arrangement of the individual sample tubes 30, 32, 33, 34 shown in the embodiment leads to a small overall size of the test kit.

The support apparatus 2 also has the two cap holders 201, 202 and the sample holder 203, which are formed as depressions on the upper side 18 of the support apparatus 2. In the embodiment shown in FIG. 2, cap holders 201, 202 and sample holder 203 are arranged in a row above the fixing devices 20, 21, 22, 23.

Sample tubes 30 contain the sample receiving device 6 in the form of a cotton swab that can be easily removed by opening the cap 40.

The sample tube 32 contains the buffer solution 7. It is provided that the sample tube 32 be placed in the sample holder 203 after being removed from the fixing device 21. Thus, the user of the test kit 1 has his or her hands free when performing the detection reaction.

Acetylcholinesterase is located in the reagent cap 42 as the first active component, and Ellman's reagent and acetylthiocholine are located in the reagent cap 43 as the second active component. Both active components are present in solid form. The tubes 33, 34 serve only for shielding the active components in the caps 43, 44 such that they cannot become contaminated or fall out. In the embodiment shown, the tubes 33, 34 also contain desiccants 50, 51 in bag form.

The cap holders 201, 202 are used to prepare the performance of the detection reactions and/or to deposit the empty caps after the active components have been dissolved out.

In the embodiment shown in FIG. 2, the individual components or the sample tubes 30, 32, 33, 34 are arranged from left to right in the sequence according to FIG. 1 that is required for carrying out the detection. This facilitates the performance of the detection and serves to minimize user errors.

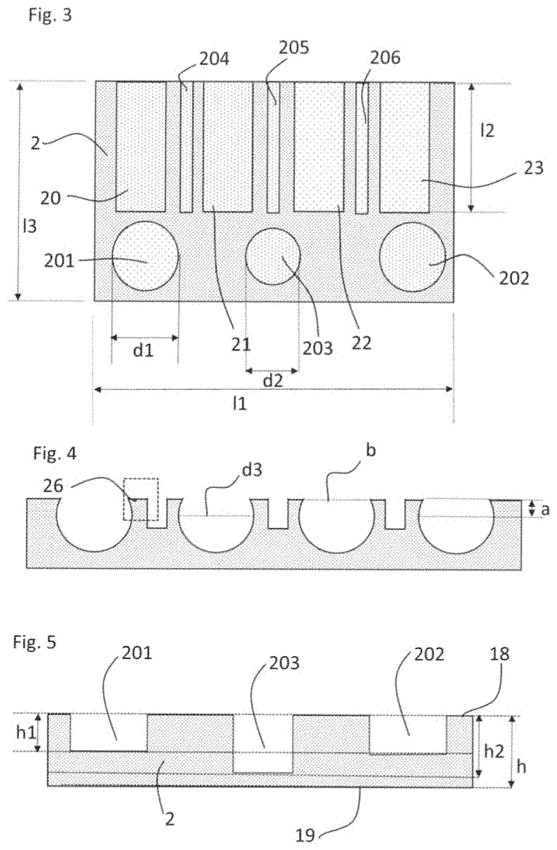

FIG. 3 is a schematic representation of the support apparatus 2 already shown in FIG. 2. In the embodiment shown here, the fixing devices are designed as a clamping device and comprise the depressions 20, 21, 22, 23 and the recesses 204, 205, 206. The depressions 20, 21, 22, 23 serve to receive the sample tubes 30, 32, 33, 34 and have a profile in the shape of a circular arc.

In the embodiment shown, the support apparatus has a length 11 of 80 mm and a length 13 of 50 mm, and is thus very compact. The diameter d1 of the cap holders 201, 202 is 15 mm, and the diameter d2 has a diameter of 12 mm. Thus, the support apparatus is particularly suitable for sample tubes having a diameter in the range of 10 to 12 mm.

FIG. 4 is a schematic representation of a cross-section through the fixing device of a support apparatus 2 as shown in FIG. 3. In this case, the chord length b of the circular arc is smaller than the diameter of the circle d3, and the arc length is greater than half the circumference. Due to this profile, the sample tubes can be fixed with corresponding diameters by clamping the sample tubes. Through the recesses 204, 205, 206 between the depressions 20, 21, 22, 23, clamping wings 26 are formed, which ensures the flexibility of the fixing device required for clamping the sample tube. The length 12 of the depressions 20, 21, 22, 23 is preferably smaller than the length of the sample tubes 30, 32, 33, 34. In this case, the diameter of the sample tubes corresponds to the diameter d3 or is slightly smaller.

FIG. 5 is a schematic representation of a cross-section through a partial region of a support apparatus 2 as shown in FIG. 3. In this case, the partial region shown in FIG. 5 comprises the cap holders 201 and 202 and the sample holder 203. Both cap holders 201, 202 and the sample holder 203 are formed by recesses in the upper side 18 of the support apparatus 2. In the embodiment shown in FIG. 5, the cap holders 201, 202 have a depth h1, and the sample holder 203 has a depth h2, wherein the depth h2 is greater than the depth h1. The cap holders 201, 202 are thus flatter than the sample holder 203. In other words, the sample holder 203 is preferably designed with a greater depth than the cap holders 201, 202—in particular, also in order to ensure secure reception of the sample tube in the receiving device. Both the cap holders 201, 202 and the sample holder 203 have a depth that is less than the thickness h of the support apparatus. Thus, the embodiment shown in FIGS. 3 through 5 has a closed underside. This is advantageous because both the cap and the sample tube are thus fixed independently of the respective substrate on which the support apparatus 2 is placed.

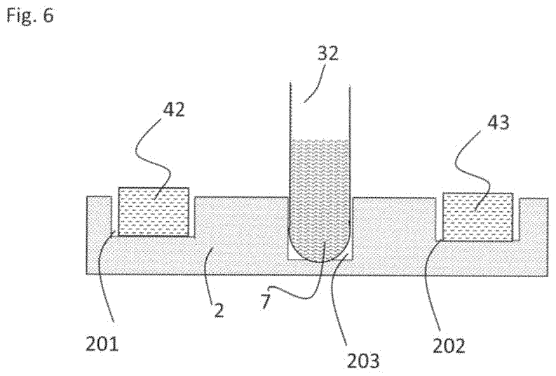

FIG. 6 is a schematic representation of the cutout shown in FIG. 5, in which the caps 42, 43 were positioned in the cap holders 201 and 202. The sample tube 30 with the buffer solution 7 is inserted in the sample holder 203.

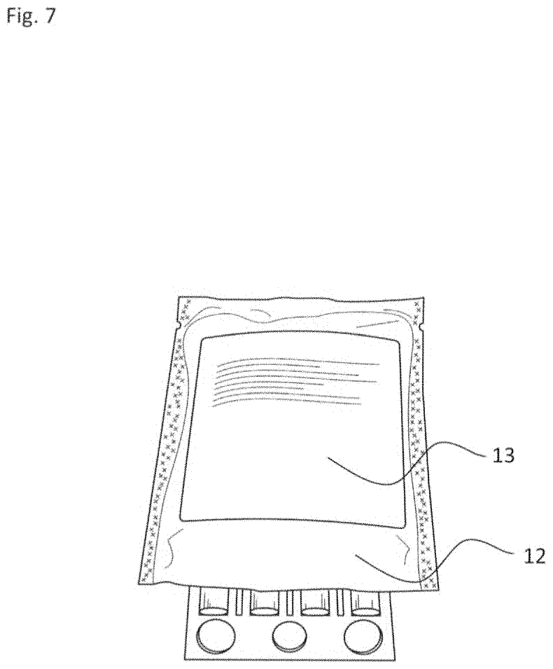

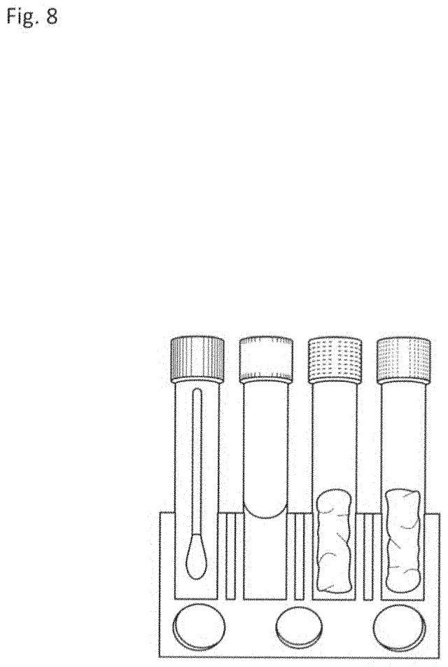

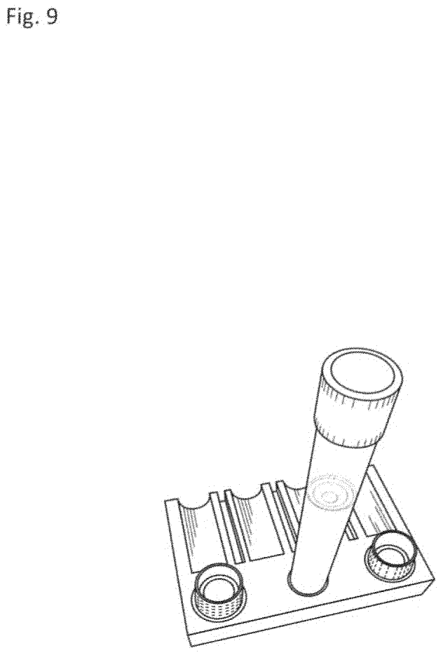

FIG. 7 shows a photograph of an embodiment in which the test kit 1 is packaged in an outer film packaging 12. By using the support apparatus 2 according to the invention, the test kit 1 can be packaged flat. On the outer packaging 12, a label 13 is expediently located on one side (front or rear side), which label contains a brief description and an instruction diagram for performing the test. A label having the relevant information (e.g., designation, production, batch no., durability) and/or the color scale is expediently located on the other side of the outer packaging 12 (consequently, not visible in FIG. 7). Because the test kit is flat, the labels can easily be read before the test kit is removed from the outer packaging.

FIG. 8 is a photograph showing the state of the test kit 1 after removal from the outer packaging. By fixing the individual sample tubes, not only can the dimensions of the test kit be minimized, but the individual components of the test kit are also presented to the user in a very clear manner. Changing the components is also avoided by the fixing of the individual components by the support apparatus. In the embodiment shown in FIG. 8, the individual components are again arranged, corresponding to FIG. 2, from left to right in the order required for the test. This also simplifies use and minimizes user errors.

FIG. 9 shows a photograph of the embodiment shown in FIG. 8 in which the individual components were arranged in the support apparatus in preparation for the performance of the test. The reagent caps having the active components acetylcholinesterase, DTNB, and acetylthiocholine were placed in the cap holders of the support apparatus. The sample tube was inserted into the sample holder. The user can thus remove the sample with the sampling device and concentrate on performing the detection reaction without searching for and/or unpacking the individual components between the individual steps.

Because the support apparatus forms secure storage for sample tubes and the caps having the active components, contamination or spillage can be avoided—particularly in the case of turbulent use environments.

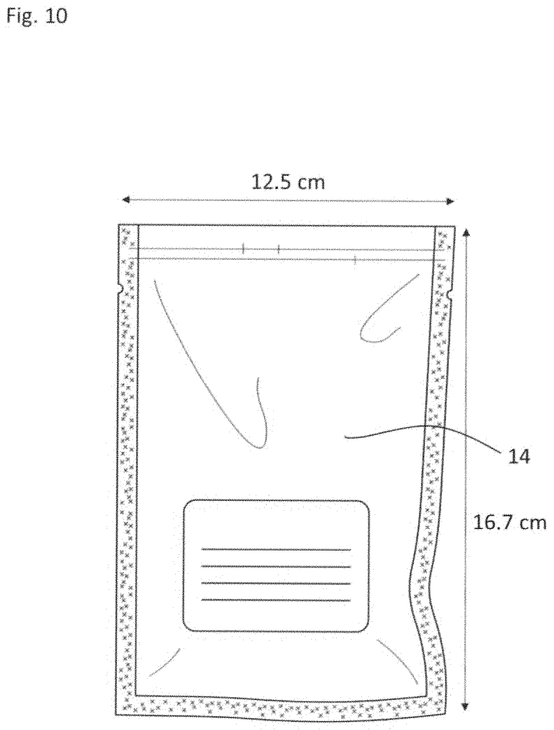

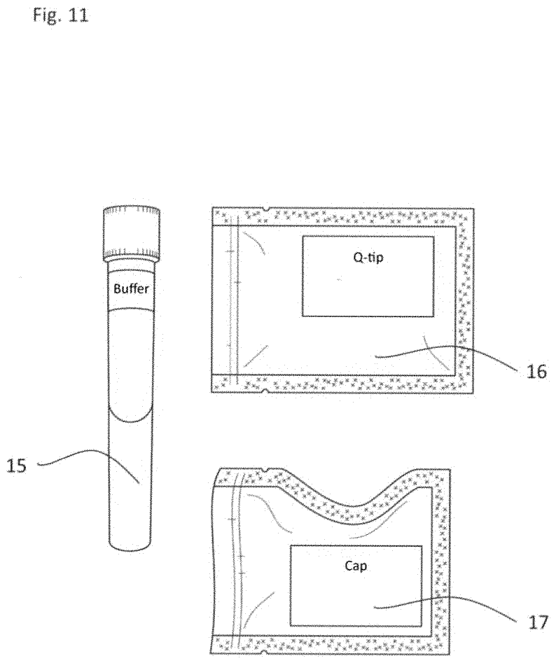

FIGS. 10 and 11 show photographic images of a known test kit as a comparison. FIG. 10 shows the test kit in an outer film packaging 14. As a result of the loose arrangement of the individual components, the outer packaging 14 has a distinct curvature. FIG. 11 shows the individual components, consisting of sample tubes having a buffer solution 15, a shrink-wrapped sampling device 16, and a film-heat-sealed outer packaging 17 that contains the reagent caps having the active components acetylcholinesterase, DTNB, and acetylthiocholine.

### LIST OF REFERENCE NUMERALS

1 test kit
2 carrier
6 sampling device
7 buffer solution
8 sample
10 sample solution having acetylcholinesterase
11 test solution having the sample solution 10, 5,5′-dithiobis(2-nitrobenzoic acid) and acetylthiocholine
12 outer packaging
13 label
14 outer packaging comparative example
15 test solution of the comparative example
16 shrink-wrapped sampling device of the comparative example
17 shrink-wrapped cap of the comparative example
18 upper side of the support apparatus
19 underside of the support apparatus
20, 21, 22, 23 fixing device
30, 32, 33, 34 sample tube
40, 41 cap
42 reagent cap having acetylcholinesterase
43 reagent cap having 5,5′-dithiobis(2-nitrobenzoic acid) and acetylthiocholine
50, 51 desiccant
201, 202 receiving device for reagent cap
203 sample holder
204, 205, 206 recess

1. A test kit for detecting organophosphate compounds, comprising: a support apparatus having an upper side and an underside, a sampling device, a buffer solution in a sample tube having a cap, and an acetylcholinesterase as the first active component, and a mixture of 5,5′-dithiobis(2-nitrobenzoic acid) (DTNB) and acetylthiocholine, wherein the sampling device is packaged in a sample tube having a cap, the first active component is applied in a first reagent cap and the second active component is applied in a second reagent cap, and the first and second reagent caps each close a sample tube, wherein the support apparatus has at least four fixing devices for receiving the sample tubes, wherein the fixing devices are formed by recesses in the support apparatus, the sample tubes are fixed parallel to the upper side of the support apparatus, and wherein the support apparatus has at least two receiving devices for receiving the first and the second reagent caps and a receiving device for vertically receiving a sample tube.

2. The test kit according to claim 1, wherein the support apparatus contains plastic, and preferably a plastic, such as acrylonitrile butadiene styrene (ABS), polylactic acid (PLA), polypropylene (PP), polystyrene (PS), polycarbonate (PC), or polyethylene (PE).

3. The test kit according to claim 1, wherein the support apparatus is designed in one piece.

4. The test kit according to claim 1, wherein the fixing devices for receiving the sample tubes are formed as depressions in the upper side of the support apparatus, and the individual fixing devices are arranged in a row on the support apparatus.

5. The test kit according to claim 1, wherein the fixing devices are designed as clamping or plug-in devices.

6. The test kit according to claim 5, wherein the fixing devices are designed as clamping devices, have a circular arc-shaped cross-section, and the arc length of the cross-section is greater than half the circumference of the corresponding circle.

7. The test kit according to claim 5, wherein the support apparatus has recesses on the upper side between the individual clamping devices, and the recesses have an angular, round, or a rectangular, cross-section.

8. The test kit according to claim 1, wherein the receiving devices for receiving the reagent caps are formed as depressions on the upper side of the support apparatus and are designed to be circular, wherein the circular diameter is larger than the diameter of the reagent caps.

9. The test kit according to claim 8, wherein a depth of the depressions is smaller than the height of the reagent caps.

10. The test kit according to claim 1, wherein the device for receiving the test tube is formed as a circular depression on the upper side of the support apparatus.

11. The test kit according to claim 1, wherein the device for receiving the test tube and the devices for receiving the reagent caps are arranged in a row,
and/or
wherein the support apparatus has a closed underside,
and/or
wherein buffer solution has a pH range of 6 to 8,
and/or
wherein the acetylcholinesterase is human acetylcholinesterase.

12. The test kit according to claim 1, wherein the test kit is shrink-wrapped in a film pouch as packaging, and/or at least one label is applied to the packaging.

13. The test kit according to claim 1, wherein the packaged test kit is packaged flat,
and/or
wherein the sample tubes that are closed with the caps contain a desiccant or a packaged desiccant,
and/or
wherein the sample tubes have a screw thread,
and/or
wherein the caps are formed as a screw cap.

14. The test kit according to claim 1, comprising a configuration to detect an acetylcholinesterase inhibitor.

15. The test kit according to claim 14, wherein the acetylcholinesterase inhibitor comprises a nerve agent.

16. The test kit according to claim 15, wherein the nerve agent comprises sarin, tabun, VX, mustard gas, soman, cyclosarin and/or Novichok, a pesticide, or another substance that inhibits acetylcholinesterase.

17. A method for detecting an acetylcholinesterase inhibitor using a test kit according to claim 1, comprising:
a) swabbing the surface to be examined using a sampling device;
b) placing the sampling device in a sample tube, wherein the sample tube contains a buffer solution; and
c) sealing the sample tube with a first reagent cap and shaking the sample tube sealed with the reagent cap, wherein acetylcholinesterase is located in the reagent cap as a first reactive component and, by shaking, any AChE inhibitor present from the sampling device and the acetylcholinesterase contained in the reagent cap are brought into the buffer solution so that a sample solution is formed.

18. The method according to claim 17, further comprising:
d) leaving the sample tube in order to binding of the Ache inhibitor possibly contained in the sample solution to the acetylcholinesterase;
e) replacing the reagent cap with a second reagent cap, the second reagent cap containing, as a second component, DTNB and acetylthiocholine, and sealing the sample tube with the second reagent cap;
f) shaking the sample tube sealed with the second reagent cap to release the second reactive component from the second reagent cap such that a solution is obtained; and
g) visually comparing a coloration of the solution with a color chart, wherein if the solution is colorless, the presence of an acetylcholinesterase inhibitor is detected.
